(19) United States
(12) Patent Application Publication
(10) Pub. No.: US 2015/0157217 A1
(43) Pub. Date: Jun. 11, 2015

(54) Analysis System for Cardiac Information and Analyzing Method Thereof

(71) Applicant: Kuo-Yuan CHANG, New Taipei City (TW)

(72) Inventor: Kuo-Yuan CHANG, New Taipei City (TW)

(21) Appl. No.: 14/475,156

(22) Filed: Sep. 2, 2014

(30) Foreign Application Priority Data
Dec. 10, 2013 (TW) 102145424

(51) Int. Cl. A61B 5/0225; A61B 5/00; A61B 5/04; A61B 5/145; A61B 5/0205; A61B 5/022; A61B 5/021; A61B 5/0402

(52) U.S. Cl. CPC A61B 5/0225; A61B 5/02108; A61B 5/7203; A61B 5/7257; A61B 5/0402; A61B 5/14542; A61B 5/0205; A61B 5/02233; A61B 5/04012

(57) ABSTRACT

The invention provides an analysis system for cardiac information and analyzing method thereof used for analyzing the cardiac information of a subject. The analysis system comprises a blood pressure measuring module connected to a cuff provided on a testing portion of the subject, used for measuring blood pressure through inflating and deflating the cuff; a computing module electrically connected to the blood pressure measuring module, used for measuring a maximum of fixed pressure for the blood pressure pulse on the testing portion; a controlling module electrically connected to the cuff and computing module, used for controlling the inflating of the cuff according to the measured fixed pressure for the blood pressure pulse; a pulse measuring module electrically connected to the controlling module; and a signal processing module electrically connected to the pulse measuring module. The invention provides a simple operating method. Not only the blood pressure can be measured, but also abnormal heart beat rate and abnormal coronary artery frequency can be analyzed. Thus abnormal structures of heart and coronary artery can be detected.

### BACKGROUND OF THE INVENTION

1. Field of the Invention

The invention relates to an analysis system for cardiac information, particularly to an analysis system for cardiac information through analyzing the heart spectrum and an analyzing method thereof.

2. Description of the Prior Art

Basic physiological signals of patients, such as blood pressure, heart beat rate, respiration number, blood oxygen, body temperature, ECG, urination, defecation, pain score, eating amount, intravenous fluid amount, etc. in hospital need to be monitored. Conventionally the data are recorded manually on medical records of patients. Modern hospitals use electric medical records. Electric monitoring system automatically transfers the monitored data to the electric medical records. However, most of the physiological signals need to be observed by eyes, written by hand, and then input to computer by nurses. It is a huge burden on manpower and time of nurses. The nurses may make record errors due to being busy on work and shifting. The difference between time shown on the instruments and unified hospital time may also cause an erroneous medication and therapy. As the patients discharge from the hospital, a small-sized electric medical instrument can provide basic physiological signals such as blood pressure and heart beat rate. There is also a need for a remote electric monitoring system, which accommodates the caring system in the hospital to remotely tracking the patients, so that caregiver can record health condition of the discharged patients and transfer the heath condition to the system.

ECG is broadly used for diagnosing heart diseases. It is particularly useful in diagnosing heart diseases such as abnormal heart rhythm and myocardium infarction. However, the principle of ECG is recording electric physiological signals. During the process, many electrodes are attached on body of a subject to measure an electric potential variation of heart. The electric physiological signals of heart don't reflect pumping ability of heart. If structures of the subject's heart or heart valves are abnormal so that the pumping ability is not complete or the heart valves can not be completely closed, the defects can't be diagnosed only through ECG. At this time, detecting whether the structures of the subject's heart or heart valves are abnormal is done through heart ultrasonic diagnosing. The principle of heart ultrasonic is observing direction and velocity of blood flow output from heart of Doppler blood flow images shown on heart ultrasonic instrument. Determine whether the structures of heart valves and coronary artery are abnormal by direction and velocity of blood flow in the image.

The subject needs to go to hospital to take abovementioned ECG and heart ultrasonic diagnosing done by medical staffs. The measured ECG and heart ultrasonic images need to be diagnosed by doctors. The subject can't do that by themselves at home. Also, the subject needs to register in advance and wait in a line. It costs more and spends more time.

### SUMMARY OF THE INVENTION

The invention provides an analysis system for cardiac information to analyze the cardiac information of a subject. The analysis system includes a blood pressure measuring module connected to a cuff provided on a testing portion of the subject, used for measuring a systolic blood pressure and a diastolic pressure through inflating and deflating the cuff; a computing module electrically connected to the blood pressure measuring module, used for measuring a maximum of fixed pressure for the blood pressure pulse at the testing portion; a controlling module electrically connected to the cuff and computing module, used for controlling the inflating of the cuff according to the measured maximum of the fixed pressure for the blood pressure pulse, and keeping the testing portion at the fixed pressure for the blood pressure pulse; a pulse measuring module electrically connected to the controlling module, used for measuring a pulse signal for a period when the testing portion is under the fixed pressure for the blood pressure pulse; and a signal processing module electrically connected to the pulse measuring module, used for computing the pulse signal to obtain a blood pressure pulse diagram to analyze the cardiac information.

The invention provides an analyzing method for analyzing cardiac information of a subject. The method includes following steps: providing a cuff on a testing portion of the subject; measuring a systolic blood pressure and a diastolic pressure of the subject; inflating the cuff according to a maximum of a fixed pressure for the blood pressure pulse on the testing portion, measuring a pulse signal for a period when the testing portion is under the maximum of the fixed pressure; and obtaining a blood pressure pulse diagram to analyze the cardiac information.

In one embodiment of the invention, the testing portion is a position where pulse of human beings can be detected, such as brachial artery or radial artery.

In one embodiment of the invention, FFT can be used to analyze the pulse signal to compute a heart spectrum and a heart noise index.

In one embodiment of the invention, the pulse signal measured when the valve of the main artery of the heart are closed and then blood flowing into the coronary artery of the heart are analyzed, and FFT is used to analyze the pulse signal to compute a heart spectrum and obtain the cardiac information of the coronary artery.

In one embodiment of the invention, ECG and blood oxygen instrument are connected and provided simultaneously on a second testing portion of the subject to analyze ECG information or blood oxygen information. Further, FFT is used to analyze the pulse signal, ECG information and blood oxygen information to obtain one or more heart spectrums to analyze the cardiac information.

In one embodiment of the invention, several cuffs are connected to several testing portions of the subject, and several pulse signals are simultaneously analyzed to test conditions of blood vessels on different testing portions.

The invention further provides an analysis system for cardiac information to analyze the cardiac information of a subject. The analysis system comprises a blood pressure measuring module connected to a cuff provided on a testing portion of the subject, used for measuring a pulse signal through inflating and deflating the cuff; an ECG module used for measuring ECG information of a second testing portion of the subject through an electric signal sensor; and a signal processing module electrically connected to the blood pressure measuring module and the ECG module, used for analyzing the pulse signal or the ECG information through FFT to compute one or more heart spectrums to analyze the cardiac information.

In one embodiment of the invention, the testing portion is a position where pulse of human beings can be detected, such as brachial artery or radial artery.

In one embodiment of the invention, FFT can be used to analyze the pulse signal to compute a heart spectrum and a heart noise index.

In one embodiment of the invention, the pulse signal measured when the valve of the main artery of the heart are closed and then blood flowing into coronary artery of the heart are analyzed, and FFT is used to analyze the pulse signals to compute a heart spectrum and obtain the cardiac information of the coronary artery.

In one embodiment of the invention, several cuffs are connected to several testing portions of the subject, and several pulse signals are simultaneously analyzed to test conditions of blood vessels on different testing portions.

In one embodiment of the invention, pulse signals of a human being are measured and transformed to a spectrum to obtain a heart spectrum. Use FFT algorithm to transform the pulse signals to frequency domain. The spectrum generally includes 3 to 5 main frequency waveforms. Peak of a first main frequency waveform is heart beat frequency. There is no other waveform outside the main frequency waveforms in a normal condition (i.e., the heart noise index is 0). If several random frequency waveforms exist outside the main frequency waveforms (i.e., the heart noise index is larger than 0), the heart condition is considered as an irregular pulse and an abnormal heart condition. So the heart spectrum and the heart noise index can be used for determining the condition of heart. Related techniques are published in inventor's Taiwan patent No. 1280119. The details are omitted herein.

In one embodiment of the invention, nutrition is provided to the heart via coronary artery. Blood is pumped out from an injunction between the main artery and heart. Valve of the main artery closes, and blood flows into the coronary artery to form capillaries in the heart, where nutrition and gases are exchanged. Then the blood flows into the coronary vein and flows out the coronary sinus formed by the coronary vein and flows into the right atrium directly. When a branch of the coronary artery is blocked, a cardiac muscle that is provided with nutrition by the branch will be necrotizing and stop contracting due to lack of oxygen and nutrition. If a lot of cardiac muscle cells are defective, the heart may stop beating. This is a general reason for heart attack. Analyzing the pulse signal when the valve of the main artery of the heart being closed and then blood flowing into the coronary artery of the heart will obtain the cardiac information of the coronary artery. Then the cardiac information can be provided to professional staffs or a doctor to determine a condition of the coronary artery of the heart of a subject.

In one embodiment of the invention, several cuffs are provided on several testing portions of the subject, such as left hand, right hand, left leg, or right leg, to analyze several pulse signals simultaneously. Thus several vessel pulse signals are obtained to test conditions of blood vessels on different testing portions.

The analysis system for analyzing cardiac information and an analyzing method thereof of the invention provides a simple analyzing and operating way, such that the subject can examine himself or herself at home without medical staffs. Compared with a conventional hemodynamometer, not only blood pressure can be measured, but also abnormal frequencies of heart beat or coronary artery can be detected. Thus whether a structure of the heart or coronary artery is normal can be determined. The analysis of the cardiac information can be a reference of clinical diagnosis. Also, users can frequently examine themselves at home to manage health conditions.

### BRIEF DESCRIPTION OF THE DRAWINGS

FIG. 1 is a flow diagram illustrating a flow of an analyzing method for cardiac information according to one embodiment of the invention.

FIG. 2 is an illustrative diagram illustrating measuring blood pressure by auscultation according to another embodiment of the invention.

FIG. 3 is a block diagram illustrating an analysis system for cardiac information according to another embodiment of the invention.

FIG. 4 is a block diagram illustrating an analysis system for cardiac information according to still another embodiment of the invention.

FIG. 5 is an illustrative diagram illustrating a heart spectrum having a heart noise index equal to 0.

FIG. 6 is an illustrative diagram illustrating a heart spectrum having a heart noise index larger than 0.

### DESCRIPTION OF THE PREFERRED EMBODIMENT

The invention will be described in detail by embodiments accompanying drawings. The objects, techniques, features and effects will be more apparently understood.

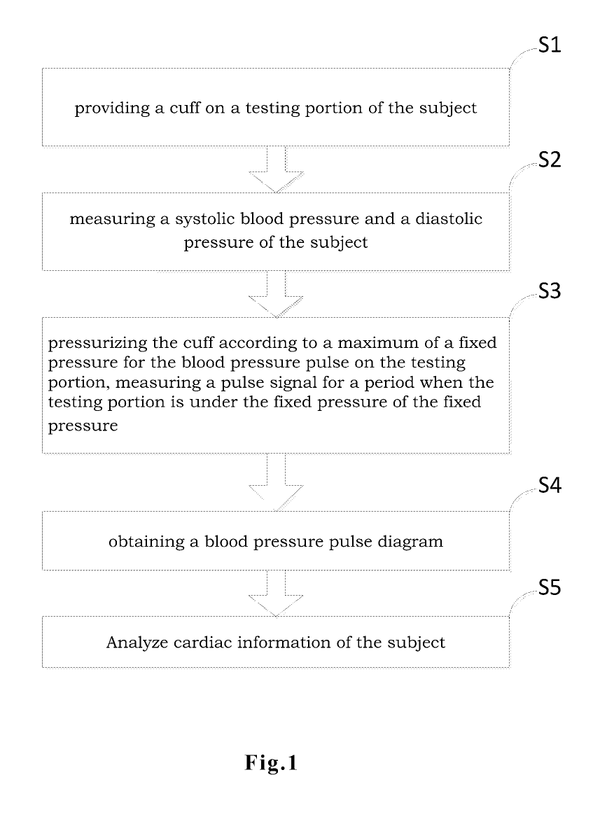

Please refer to FIG. 1 illustrating a flow of an analyzing method for cardiac information according to one embodiment of the invention. The analyzing method comprises following steps. First, provide a cuff on a testing portion of a subject for measuring a blood pressure. The pressure applied on the testing portion by the cuff may be adjusted by inflation or contracting of the cuff. Measure a systolic blood pressure and a diastolic blood pressure by oscillometric method (step S1). The principle of oscillometric method is increasing the pressure of the cuff to an extent that is a little bit more than the systolic blood pressure. At this time, the artery of the arm is blocked. Then the cuff depressurizes slowly. Until the time that blood flows through the artery of the arm, the volume of vessel changes and produces oscillated pressure signal. The oscillation of pressure signal is an unstable phenomenon produced by blood flowing through blocked point so that vessel wall vibrates accordingly. Thus the volume of vessel changes accordingly and pressure in the cuff oscillates. One who masters the test can measure the systolic blood pressure and diastolic pressure by the pressure oscillation in the cuff.

After the systolic blood pressure and diastolic pressure are measured (step S2, keep a pressure according to a maximum of a fixed pressure for the blood pressure pulse. Then contract the cuff and apply the fixed pressure on the testing portion and measure a heart pulse signal of the testing portion for a period (step S3). For example, in previous step S2, the systolic blood pressure is 120 mmHg and the diastolic pressure is 80 mmHg. After the systolic blood pressure and the diastolic pressure are measured, an in-between value of the systolic blood pressure and diastolic pressure of 90 mmHg is selected. Then proceed to step S3. The testing portion is applied to the fixed 90 mmHg pressure by inflation or contracting of the cuff. Measure the pulse signal of the testing portion for a period of 30 seconds. The time period may be varied.

According to the pulse signal, obtain a blood pulse pressure diagram (step S4) to analyze the cardiac information of the subject (step S5).

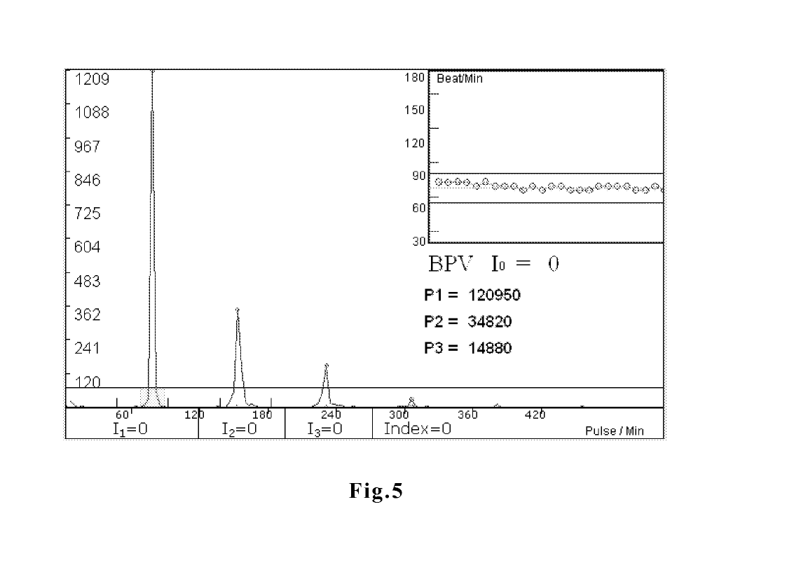

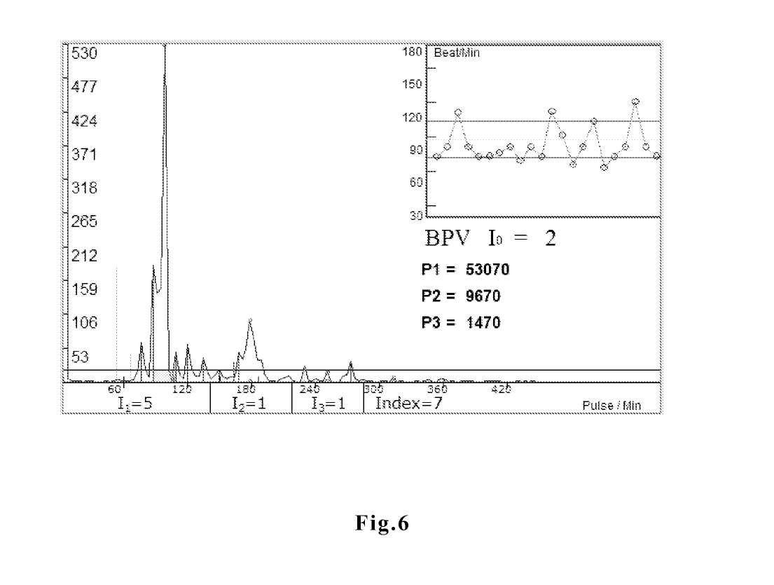

Subsequently, use FFT to transform the pulse signal lasting for the period of time to a heart spectrum. The heart beat frequency can be analyzed in frequency domain. Generally, there are several main frequency waveforms in the heart spectrum. For example, a first main frequency waveform is the heart beat frequency of human being. A second main frequency waveform is the heart valve beat frequency. A third main frequency waveform is the heart vessel beat frequency (i.e., the vibration produced by heart pumping out blood to coronary artery). As shown in FIG. 5, in a normal condition, there is no other waveform outside the main frequency waveforms (i.e., the heart noise index equals to 0). If one or more random waveforms exist outside the main frequency waveforms of the heart spectrum (i.e., the heart noise index is larger than 0), as shown in FIG. 6, it is considered as an abnormal condition of the heart beat or irregular opening/closing of the heart valves. The reason may be insufficiency or prolapse of the heart valves. Through analyzing the distribution of waveforms, except the main frequency waveforms, whether the structure of the heart is abnormal can be determined according to abnormal random waveforms existing in the heart spectrum, which represent that heart beat is abnormal.

In one embodiment of the invention, the pulse signal measured when the valves of the main artery of the heart are closed and then blood flowing into the coronary artery of the heart are analyzed, and FFT is used to analyze the pulse signal to compute a heart spectrum and obtain the cardiac information of the coronary artery.

In one embodiment of the invention, ECG and blood oxygen instrument are connected and provided simultaneously on a second testing portion of the subject to analyze ECG information or blood oxygen information. Further, FFT is used to analyze the pulse signal, ECG information or blood oxygen information to obtain one or more heart spectrums to analyze the cardiac information.

In one embodiment of the invention, several cuffs are simultaneously connected to several testing portions of the subject, and several pulse signals are simultaneously analyzed to test conditions of blood vessels on different testing portions.

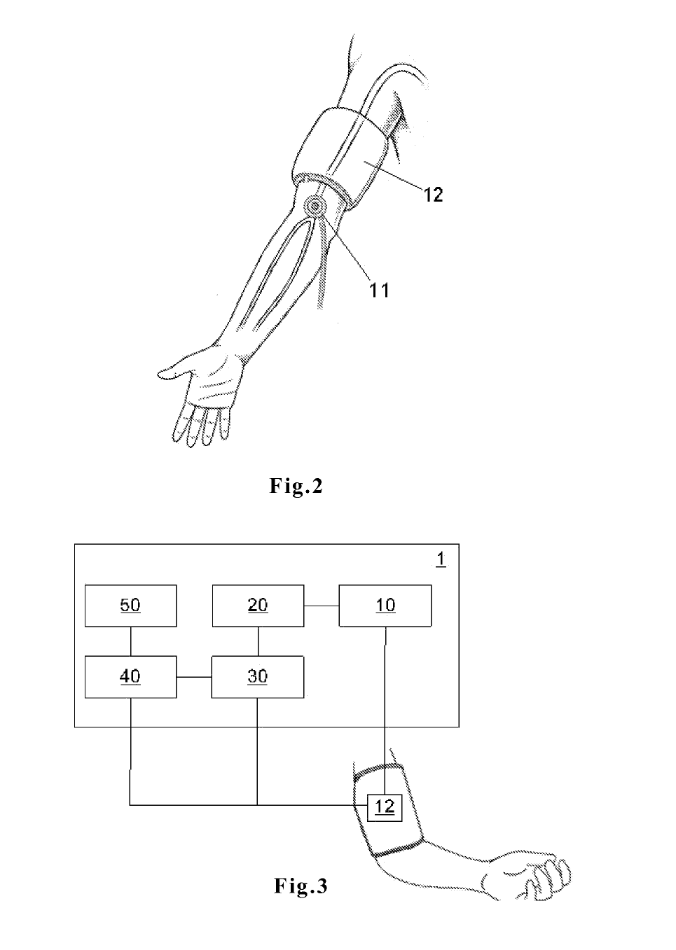

In step S1 of abovementioned embodiment, use oscillometric method to measure a systolic blood pressure and a diastolic blood pressure. The subject needs to keep his/her arm stationary to prevent measuring error caused by vibration of his/her arm. For special conditioned patients who can't keep stationary, use auscultation method to measure a systolic blood pressure and a diastolic blood pressure. As shown in FIG. 2, the cuff 12 surrounds the arm of the subject. Provide a stethoscope 11 at the artery of the arm. One who masters the test or the subject hears the korotkoff sounds by auscultation method combined with reading value of the blood pressure, then a systolic blood pressure and a diastolic blood pressure can be measured.

In one embodiment of the invention, in step S3, the computing equation of the average blood pressure value is BPₘₑₐₙ=⅓×SBP+⅔×DBP. BPₘₑₐₙ is an average blood pressure value (a maximum of fixed pressure for the blood pressure pulse). SBP is the systolic blood pressure. DBP is the diastolic blood pressure.

Refer to FIG. 3 illustrating an analysis system for cardiac information according to another embodiment of the invention. The analysis system 1 comprises a blood pressure measuring module 10 connected to a cuff 12 provided on a testing portion of the subject, used for measuring a systolic blood pressure and a diastolic pressure of the testing portion through inflating and deflating the cuff 12; a computing module 20 electrically connected to the blood pressure measuring module 10, used for measuring a maximum of fixed pressure for the blood pressure pulse at the testing portion; a controlling module 30 electrically connected to cuff 12 and computing module 20, used for controlling the inflating of cuff 12 according to the measured fixed pressure for the blood pressure pulse, and keeping the testing portion under the fixed pressure for the blood pressure pulse; a pulse measuring module 40 electrically connected to the controlling module 30, used for measuring a pulse signal for a period when the testing portion is under the fixed pressure for the blood pressure pulse; and a signal processing module 50 electrically connected to the pulse measuring module 40, used for computing the pulse signal to obtain a blood pressure pulse diagram to analyze the cardiac information.

In one embodiment of the invention, the testing portion is a position where pulse of human beings can be detected, such as brachial artery or radial artery.

In one embodiment of the invention, FFT can be used to analyze the pulse signal to compute a heart spectrum and a heart noise index.

In one embodiment of the invention, the pulse signal measured when the valves of the main artery of the heart are closed and then blood flowing into the coronary artery of the heart are analyzed, and FFT is used to analyze the pulse signal to compute a heart spectrum and obtain the cardiac information of the coronary artery.

In one embodiment of the invention, ECG and blood oxygen instrument are connected and provided simultaneously to a second testing portion of the subject to analyze ECG information or blood oxygen information. Further, FFT is used to analyze the pulse signal, ECG information or blood oxygen information to obtain one or more heart spectrums to analyze the cardiac information.

In one embodiment of the invention, several cuffs are simultaneously connected to the blood pressure measuring module 10 and to several testing portions of the subject, and several pulse signals are simultaneously analyzed to test conditions of blood vessels on different testing portions.

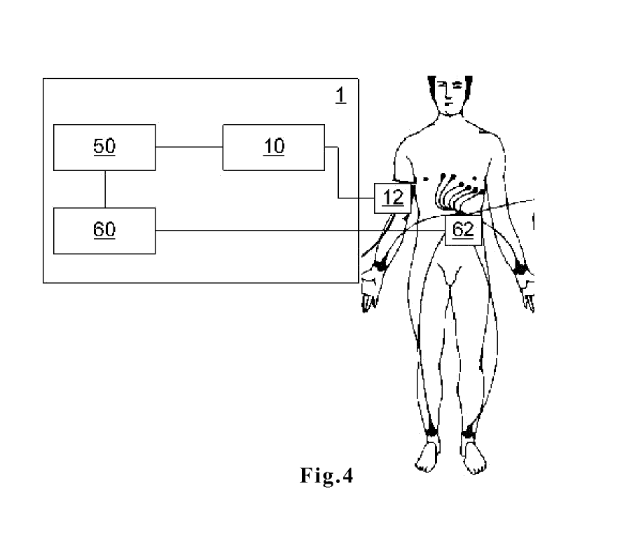

Please refer to FIG. 4. The invention further provides an analysis system 1 for cardiac information to analyze the cardiac information of a subject. The analysis system comprises a blood pressure measuring module 10 connected to a cuff 12 provided on a testing portion of the subject, used for measuring a pulse signal at the testing portion of the subject through inflating and deflating the cuff 12; an ECG module 60 used for measuring ECG information of a second testing portion of the subject through an electric signal sensor 62; and a signal processing module 50 electrically connected to the blood pressure measuring module and the ECG module, used for analyzing the pulse signal or the ECG information through FFT to compute one or more heart spectrums to analyze the cardiac information.

In one embodiment of the invention, the testing portion is a position where pulse of human beings can be detected, such as brachial artery or radial artery.

In one embodiment of the invention, FFT can be used to analyze the pulse signal to compute a heart spectrum and a heart noise index.

In one embodiment of the invention, the pulse signals measured when the valves of the main artery of the heart are closed and then blood flowing into the coronary artery of the heart are analyzed, and FFT is used to analyze the pulse signal to compute a heart spectrum and obtain the cardiac information of the coronary artery.

In one embodiment of the invention, several cuffs are simultaneously connected to the blood pressure measuring module 10 and to several testing portions of the subject, and several pulse signals are simultaneously analyzed to test conditions of blood vessels on different testing portions.

To sum up the foregoing descriptions, the analysis system for analyzing the cardiac information of the invention provides a simple analyzing and operating way, such that the subject can examine himself or herself at home without medical staffs. Compared with a conventional hemodynamometer, not only blood pressure can be measured, but also abnormal frequency of heart beat or coronary artery can be detected. Thus whether structure of the heart or coronary artery is normal can be determined. The analysis of the cardiac information can be a reference of clinical diagnosis. Also, users can frequently examine themselves at home to manage health conditions.

While the invention can be subject to various modifications and alternative forms, a specific example thereof has been shown in the drawings and is herein described in detail. It should be understood, however, that the invention is not to be limited to the particular form disclosed, but on the contrary, the invention is to cover all modifications, equivalents, and alternatives falling within the spirit and scope of the appended claims.

What is claimed is:

1. An analysis system for analyzing cardiac information of a subject, comprising:
a blood pressure measuring module, connected to a cuff provided on a testing portion of the subject, used for measuring a systolic blood pressure and a diastolic pressure of the testing portion through inflating and deflating the cuff;
a computing module, electrically connected to the blood pressure measuring module, used for measuring a maximum of a fixed pressure for the blood pressure pulse on the testing portion;
a controlling module, electrically connected to the cuff and the computing module, used for controlling the inflating of the cuff according to the measured maximum of the fixed pressure for the blood pressure pulse and keeping the testing portion at the fixed pressure for the blood pressure pulse;
a pulse measuring module, electrically connected to the controlling module, used for measuring a pulse signal for a period when the testing portion is under the fixed pressure for the blood pressure pulse; and
a signal processing module, electrically connected to the pulse measuring module, used for computing the pulse signal to obtain a blood pressure pulse diagram to analyze the cardiac information.

2. The analysis system as claim 1 further comprising using FFT to analyze the pulse signal to compute a heart spectrum and a heart noise index.

3. The analysis system as claim 1 further comprising analyzing the pulse signal when the valve of the main artery of the heart being closed and then blood flowing into the coronary artery of the heart, and using FFT to analyze the pulse signal to compute a heart spectrum and obtain the cardiac information of the coronary artery.

4. The analysis system as claim 1 further comprising connecting ECG or blood oxygen instrument simultaneously to a second testing portion of the subject to analyze ECG information or blood oxygen information, and using FFT to analyze the pulse signal, ECG information or blood oxygen information to compute and obtain one or more heart spectrums to analyze the cardiac information.

5. The analysis system as claim 1 further comprising connecting several cuffs to several testing portions of the subject, and several pulse signals being simultaneously analyzed to test conditions of blood vessels on different testing portions.

6. An analysis system for analyzing cardiac information of a subject, comprising:
a blood pressure measuring module, connected to a cuff provided on a testing portion of the subject, used for measuring a pulse signal of the testing portion through inflating and deflating the cuff;
an ECG module, used for measuring ECG information of a second testing portion of the subject through an electric signal sensor; and
a signal processing module, electrically connected to the blood pressure measuring module and the ECG module, used for analyzing the pulse signal or the ECG information through FFT to compute one or more heart spectrums to analyze the cardiac information.

7. The analysis system as claim 6 further comprising using FFT to analyze the pulse signal to compute a heart spectrum and a heart noise index.

8. The analysis system as claim 6 further comprising analyzing the pulse signal when the valve of the main artery of the heart being closed and then blood flowing into the coronary artery of the heart, and using FFT to analyze the pulse signal to compute a heart spectrum and obtain the cardiac information of the coronary artery.

9. The analysis system as claim 6 further comprising connecting several cuffs to several testing portions of the subject, and several pulse signals being simultaneously analyzed to test conditions of blood vessels on different testing portions.

10. An analyzing method for analyzing cardiac information of a subject, comprising:
providing a cuff on a testing portion of the subject;
measuring a systolic blood pressure and a diastolic pressure of the subject;
inflating the cuff according to a maximum of a fixed pressure for the blood pressure pulse on the testing portion, measuring a pulse signal for a period when the testing portion is under the maximum of the fixed pressure; and
obtaining a blood pressure pulse diagram to analyze the cardiac information.

11. The analyzing method as claim 10 further comprising using FFT to analyze the pulse signal to compute a heart spectrum and a heart noise index.

12. The analyzing method as claim 10 further comprising analyzing the pulse signal when the valve of the main artery of the heart being closed and then blood flowing into the coronary artery of the heart, and using FFT to analyze the pulse signal to compute a heart spectrum and obtain the cardiac information of the coronary artery.

13. The analyzing method as claim 10 further comprising connecting ECG or blood oxygen instrument simultaneously to a second testing portion of the subject to analyze ECG information or blood oxygen information, and using FFT to analyze the pulse signal, ECG information or blood oxygen information to compute and obtain one or more heart spectrums to analyze the cardiac information.

14. The analyzing method as claim 10 further comprising connecting several cuffs to several testing portions of the subject, and several pulse signals being simultaneously analyzed to test conditions of blood vessels on different testing portions.
